(19) United States
(12) Patent Application Publication
(10) Pub. No.: US 2005/0222094 A1
(43) Pub. Date: Oct. 6, 2005

(54) Treatment of inflammatory bowel disease

(76) Inventors: Robert M. Burk, Laguna Beach, CA (US); Mark Holoboski, Laguna Niguel, CA (US); Mari F. Posner, Laguna Niguel, CA (US); Karen M. Kedzie, Rancho Santa Margarita, CA (US); Daniel W. Gil, Corona Del Mar, CA (US)

Correspondence Address: Brent Johnson, Patent Agent (T2-7H);ALLERGAN, INC., Legal Department, 2525 Dupont Drive, Irvine, CA, 92612, US

(21) Appl. No.: 11/133,047

(22) Filed: May 18, 2005

Related U.S. Application Data
(63) Continuation-in-part of application No. 10/672,499, filed on Sep. 26, 2003.
Application No. 10/672,499 is a continuation of application No. 10/346,828, filed on Jan. 16, 2003, now Pat. No. 6,767,920.
Application No. 10/346,828 is a continuation-in-part of application No. 09/882,720, filed on Jun. 14, 2001, now abandoned.

(51) Int. Cl. A61K 31/66; A61K 31/557

(52) U.S. Cl. 514/125; 514/381; 514/573; 514/600; 514/615

(57) ABSTRACT

A method is disclosed herein comprising administering a compound to a mammal suffering from an inflammatory bowel disease for the treatment of said disease, said compound represented by the general formula I; 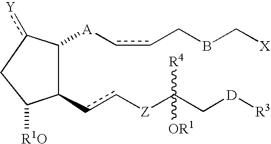
wherein A, B, D, X, Y, Z, R¹, R³ and R⁴ are as defined in the specification.

### CROSS REFERENCE TO RELATED APPLICATIONS

This patent application is a continuation in part of Ser. No. 10/672,499, filed Sep. 26, 2003, which is a continuation of Ser. No. 10/346,828, filed Jan. 16, 2003, now U.S. Pat. No. 6,767,920, which is a continuation in part of Ser. No. 09/882,720, filed Jun. 14, 2001, now abandoned.

### BACKGROUND OF THE INVENTION

1. Field of the Invention

The present invention relates to 3, 7 or 3 and 7 thia or oxa prostanoic acid derivatives for the treatment of inflammatory bowel disease.

2. Description of Related Art

Prostaglandins can be described as derivatives of prostanoic acid which have the following structural formula:

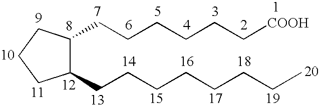

Various types of prostaglandins are known, depending on the structure and substituents carried on the alicyclic ring of the prostanoic acid skeleton. Further classification is based on the number of unsaturated bonds in the side chain indicated by numerical subscripts after the generic type of prostaglandin [e.g. prostaglandin E₁ (PGE₁), prostaglandin E₂ (PGE₂)], and on the configuration of the substituents on the alicyclic ring indicated by α or β [e.g. prostaglandin F_{2α} (PGF_{2β})].

Prostaglandins are useful for the long-term medical management of glaucoma (see, for example, Bito, L. Z. Biological Protection with Prostaglandins, Cohen, M. M., ed., Boca Raton, Fla., CRC Press Inc., 1985, pp. 231-252; and Bito, L. Z., Applied Pharmacology in the Medical Treatment of Glaucomas Drance, S. M. and Neufeld, A. H. eds., New York, Grune & Stratton, 1984, pp. 477-505. Such prostaglandins include PGF_{2α}, PGF_{1α}, PGE₂, and certain lipid-soluble esters, such as C₁ to C₂ alkyl esters, e.g. 1-isopropyl ester, of such compounds.

EP 0 985 663 A1 discloses compounds such as the one shown below.

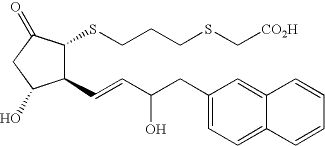

Inflammatory bowel disease (IBD) is a group of disease characterized by inflammation in the large or small intestines and is manifest in symptoms such as diarrhea, pain, and weight loss. Nonsteroidal anti-inflammatory drugs have been shown to be associated with the risk of developing IBD, and recently Kabashima and colleagues have disclosed that “EP4 works to keep mucosal integrity, to suppress the innate immunity, and to downregulate the proliferation and activation of CD4+ T cells. These findings have not only elucidated the mechanisms of IBD by NSAIDs, but also indicated the therapeutic potential of EP4-selective agonists in prevention and treatment of IBD.” (Kabashima, et. al., The Journal of Clinical Investigation, April 2002, Vol. 9, 883-893)

### SUMMARY OF THE INVENTION

Methods are disclosed herein for the treatment of inflammatory bowel disease by administration of compounds of formula I 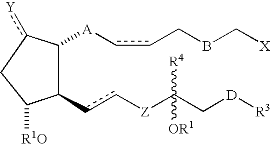
wherein hatched lines represent the α configuration, a triangle represents the β configuration, a wavy line represents either the α configuration or the β configuration and a dotted line represents the presence or absence of a double bond;
A and B are independently selected from the group consisting of O, S and CH₂;
provided that at least one of A or B is S;
D represents a covalent bond or CH₂, O, S or NH;
X is CO₂R, CONR₂, CH₂OR, P(O)(OR)₂, CONRSO₂R, SONR₂ or 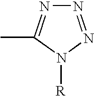
Y is O, OH, OCOR², halogen or cyano;
Z is CH₂ or a covalent bond;
R is H or R²;
R¹ is H, R², phenyl, or COR²;
R² is C₁-C₅ lower alkyl or alkenyl;
R³ is benzothienyl, benzofuranyl, naphthyl, or substituted derivatives thereof, wherein the substituents maybe selected from the group consisting of C₁-C₅ alkyl, halogen, CF₃, CN, NO₂, NR₂, CO₂R and OR; and
R⁴ is hydrogen or C₁-C₅ alkyl.
   Compositions, medicaments, and dosage forms related thereto are also disclosed.

### BRIEF DESCRIPTION OF THE DRAWING FIGURES

FIG. 1 is a schematic of the chemical synthesis of a certain intermediate for the compounds of the invention as disclosed in Examples 1 through 3.

FIG. 2 is a schematic of the chemical synthesis of certain compounds related to the compounds of the invention as disclosed in Examples 4 through 7.

### DETAILED DESCRIPTION OF THE INVENTION

Treatment of inflammatory bowel disease may be accomplished by the administration of the compounds described herein to the suffering mammal. Inflammatory bowel disease describes a variety of diseases characterized by inflammation of the bowels including, but not limited to, ulcerative colitis and Crohn's disease. Treatment may be accomplished by oral administration, by suppository, or parenteral administration, or some other suitable method.

The compounds used for the treatment are encompassed by the following structural formula I:

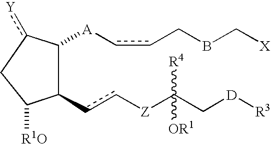

A preferred group of the compounds of the present invention includes compounds that have the following structural formula II:

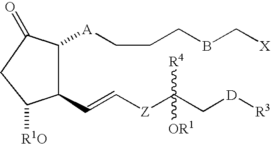

Another preferred group includes compounds having the formula III:

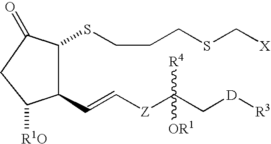

In the above formulae, the substituents and symbols are as hereinabove defined.

In the above formulae:

Preferably A and B are both S.

Preferably D represents a covalent bond or is CH₂; more preferably D is CH₂.

Preferably Z represents a covalent bond.

Preferably R is H.

Preferably R¹ is H.

Preferably R⁴ is hydrogen or methyl, most preferably hydrogen.

Preferably Y═O.

Preferably X is CO₂R and more preferably R is selected from the group consisting of H, methyl, i-propyl and n-propenyl.

The above compounds of the present invention may be prepared by methods that are known in the art or according to the working examples below. The compounds, below, are especially preferred representative, of the compounds of the present invention.
{3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid methyl ester,
{3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid,
{3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid isopropyl ester,
{3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid methyl ester,
{3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid,
{3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid isopropyl ester,
{3-[(1R,2S,3R)-3-Hydroxy-2-((S)-(E)-3-hydroxy-5-(benzofuranyl)pent-1-enyl)-5-oxocyclopentylsulfanyl]propylsulfanyl}acetic acid methyl ester,
{3-[(1R,2S,3R)-3-Hydroxy-2-((S)-(E)-3-hydroxy-5-(benzofuranyl)pent-1-enyl)-5-oxocyclopentylsulfanyl]propylsulfanyl}acetic acid,
{3-[(1R,2S,3R)-3-Hydroxy-2-((S)-(E)-3-hydroxy-5-(benzofuranyl)pent-1-enyl)-5-oxocyclopentylsulfanyl]propylsulfanyl}acetic acid isopropyl ester,
{3-[(1R,2S,3R)-3-Hydroxy-2-((E)-3-hydroxy-4-naphthalen-2-yl-but-1-enyl)-5-oxocyclopentylsulfanyl]propylsulfanyl}acetic acid methyl ester,
{3-[(1R,2S,3R)-3-Hydroxy-2-((E)-3-hydroxy-4-naphthalen-2-yl-but-1-enyl)-5-oxocyclopentylsulfanyl]propylsulfanyl}acetic acid,
{3-[(1R,2S,3R)-2-((E)-4-Benzo[b]thiophen-3-yl-3-hydroxybut-1-enyl)-3-hydroxy-5-oxocyclopentylsulfanyl]propylsulfanyl}acetic acid methyl ester,
{3-[(1R,2S,3R)-2-((E)-4-Benzo[b]thiophen-3-yl-3-hydroxybut-1-enyl)-3-hydroxy-5-oxocyclopentylsulfanyl]propylsulfanyl}acetic acid,
{3-[(1R,2S,3R)-3-Hydroxy-2-((S)-(E)-3-(hydroxy)-3-(methyl)-5-(naphthyl)pent-1-enyl)-5-oxocyclopentylsulfanyl]propylsulfanyl}acetic acid methyl ester,
{3-[(1R,2S,3R)-3-Hydroxy-2-((E)-3-hydroxy-3-methyl-4-naphthalen-2-yl-but-1-enyl)-5-oxocyclopentylsulfanyl]propylsulfanyl}acetic acid methyl ester,
{3-[(1R,2S,3R)-3-Hydroxy-2-((E)-3-hydroxy-3-methyl-4-naphthalen-2-yl-but-1-enyl)-5-oxocyclopentylsulfanyl]propylsulfanyl}acetic acid,
{3-[(1R,2S,3R)-3-Hydroxy-2-((S)-(E)-3-(hydroxy)-3-(methyl)-5-(naphthyl)but-1-enyl)-5-oxocyclopentylsulfanyl]propylsulfanyl}acetic acid,
{3-[(1R,2S,3R)-3-Hydroxy-2-((S)-(E)-3-(hydroxy)-3-(methyl)-5-(benzothienyl)pent-1-enyl)-5-oxocyclopentylsulfanyl]propylsulfanyl}acetic acid methyl ester and
{3-[(1R,2S,3R)-3-Hydroxy-2-((S)-(E)-3-(hydroxy)-3-(methyl)-5-(benzothienyl)pent-1-enyl)-5-oxocyclopentylsulfanyl]propylsulfanyl}acetic acid methyl ester.

Those skilled in the art will readily understand that for oral or rectal administration the compounds of the invention are admixed with pharmaceutically acceptable excipients which per se are well known in the art. Specifically, a drug to be administered systemically, it may be confected as a powder, pill, tablet or the like, or as a syrup or elixir suitable for oral administration. Description of the substances normally used to prepare tablets, powders, pills, syrups and elixirs can be found in several books and treatise well known in the art, for example in Remington's Pharmaceutical science, Edition 17, Mack Publishing Company, Easton, Pa.

Parenteral administration is generally characterized by injection. Injectables can be prepared in conventional forms, either as liquid solutions or suspensions, solid forms suitable for dissolving or suspending in liquid prior to injection, or as emulsions. Descriptions of substances and methods normally used to prepare formulations for parenteral administration can be found in several treatises and books well known in the art such as, Handbook On Injectable Drugs (11th edition), edited by Lawrence A. Trissel, (Chicago: Login Brothers Book Company; Jan. 15, 2001).

Treatment of inflammatory bowel disease may be accomplished by the administration of the compounds described herein to the suffering mammal. Inflammatory bowel disease describes a variety of diseases characterized by inflammation of the bowels including, but not limited to, ulcerative colitis and Crohn's disease. Treatment may be accomplished by oral administration, by suppository, or parenteral administration, or some other suitable method.

While not intending to limit the scope of the invention in any way, delivery of the compounds disclosed herein to the colon via oral dosage forms may be accomplished by any of a number of methods known in the art. For example, reviews by Chourasia and Jain in J Pharm Pharmaceut Sci 6 (1): 33-66, 2003 and Shareef et. al (AAPS PharmSci 2003; 5 (2) Article 17) describe a number of useful methods. While not intending to limit the scope of the invention in any way these methods include 1) administration of a prodrug, including an azo or a carbohydrate based prodrug; 2) coating the drug with, or encapsulating or impregnating the drug into a polymer designed for delivery to the colon, 3) time released delivery of the drug, 4) use of a bioadhesive system; and the like.

While not intending to be bound in any way by theory, it is believed that intestinal microflora are capable of reductive cleavage of an azo bond leaving the two nitrogen atoms as amine functional groups. While not intending to limit the scope of the invention in any way, the azo prodrug approach has been used to deliver to 5-aminosalicylic acid to the colons of humans in clinical trials for the treatment of inflammatory bowel disease. It is also believed that bacteria of the lower GI also have enzymes which can digest glycosides, glucuronides, cyclodextrins, dextrans, and other carbohydrates, and ester prodrugs formed from these carbohydrates have been shown to deliver the parent active drugs selectively to the colon. For example, in vivo and in vitro studies on rats and guinea pigs with prodrugs of dexamethasone, prednisolone, hydrocortisone, and fludrocortisone, suggest that glycoside conjugates may be useful for the delivery of steroids to the human colon. Other in vivo studies have suggested that glucouronide, cyclodextrin, and dextran prodrugs of steroids or non-steroidal anti-inflammatory drugs are useful for delivery of these drugs to the lower GI tract. An amide of salicylic acid and glutamic acid has been shown to be useful for the delivery of salicylic acid to the colon of rabbit and dog.

While not intending to limit the scope of the invention in any way, carbohydrate polymers such as amylase, arabinogalactan, chitosan, chondroiton sulfate, dextran, guar gum, pectin, xylin, and the like, or azo-group containing polymers can be used to coat a drug compound, or a drug may be impregnated or encapsulated in the polymer. It is believed that after oral administration, the polymers remain stable in the upper GI tract, but are digested by the microflora of the lower GI thus releasing the drug for treatment.

Polymers which are sensitive to pH may also be used since the colon has a higher pH than the upper GI tract. Such polymers are commercially available. For example, Rohm Pharmaceuticals, Darmstadt, Germany, markets pH dependent methacrylate based polymers and copolymers which have varying solubilities over different pH ranges based upon the number of free carboxylate groups in the polymer under the tradename Eudragit®. Several Eudragit® dosage forms are currently used to deliver salsalazine for the treatment of ulcerative colitis and Crohn's disease. Time release systems, bioadhesive systems, and other delivery systems have also been studied. For the treatment of inflammatory bowel disease, combination treatment with the following classes of drugs are contemplated:
aminosalicylates including sulfasalazaline, mesalazine, sulfasalazine, mesalamine, Olsalazine, balsalazide, and the like;
corticosteroids including methotrexate, cortisone, hydrocortisone, prednisone, prednisolone, methylprednisone, triamcinolone, fluoromethalone, dexamethasone, medrysone, betamethasone, loteprednol, fluocinolone, flumethasone, mometasone, and the like;
immunomodulators including azathioprine, 6-mercaptopurine, cyclosporine, and the like; and
inhibitors of inflammatory cytokines (TNF) such as inflixitmab, CDP571, CDP870, etanercept, onercept, adalimumab, and the like.

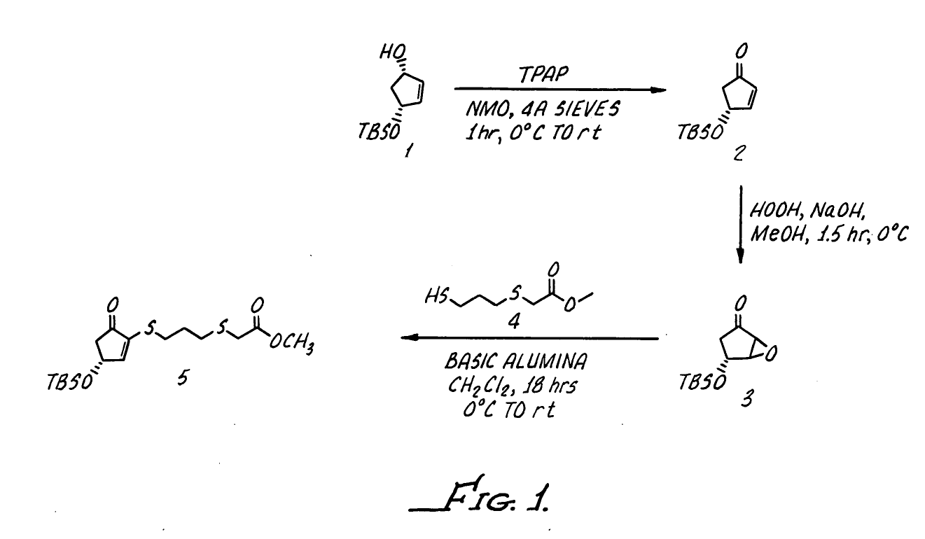

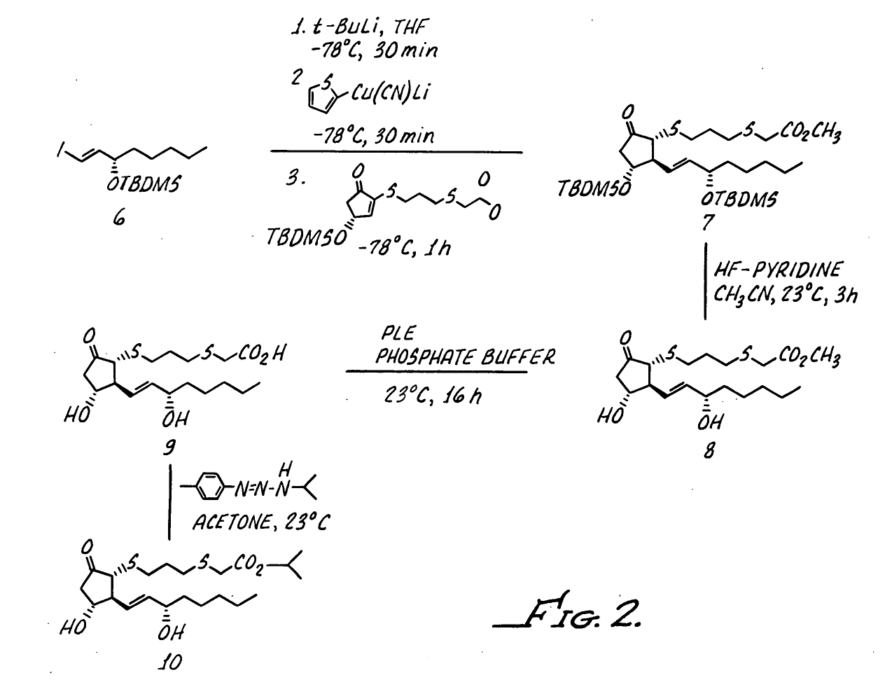

The invention is further illustrated by the following non-limiting Examples, which are summarized in the reaction schemes of FIGS. 1 and 2 wherein the compounds are identified by the same designator in both the Examples and the Figures.

### EXAMPLE 1

### (R)-4-(tert-Butyldimethylsilanyloxy)cyclopent-2-enone (2)

Tetrapropylammonium perruthenate (9.4 mg, 0.027 mmol) was added to a mixture of (1S,4R)-4-(tert-butyldimethylsilanyloxy)cyclopent-2-enol prepared, according to Tetrahedron Letters, Vol. 37, No. 18, 1996, pp. 3083-6, (118.6 mg, 0.54 mmol), 4-methylmorpholine N-oxide (94.9 mg, 0.81 mmol) and crushed 4 Å sieves (270 mg) in CH₂Cl₂ (10 mL). The mixture was stirred for 30 min and was passed through a plug of silica gel with CH₂Cl₂. The filtrate was concentrated in vacuo to give 100 mg (86%) of the above titled compound.

### EXAMPLE 2

### (R)-4-(tert-Butyldimethylsilanyloxy)-6-oxabicyclo [3.1.0]hexan-2-one (3)

Hydrogen peroxide (4.5 mL, 46.3 mmol, 30% wt. % solution in water) and 1N NaOH (46 μL, 0.046 mmol) were added to a solution of enone 2 (2.5 g, 11.5 mmol) in MeOH (30 mL) at 0° C. After stirring 1.5 h at 0° C. the mixture was concentrated in vacuo, washed with saturated aqueous NH₄Cl and extracted with CH₂Cl₂ (3×). The combined organics were washed with brine, dried (Na₂SO₄), filtered and concentrated in vacuo to afford the above titled compound.

### EXAMPLE 3

### ({3-[(R)-3-(tert-Butyldimethylsilanyloxy)-5-oxocyclopent-1-enylsulfanyl]propylsulfanyl}acetic acid methyl ester (5)

The epoxide 3 prepared above was diluted with CH₂Cl₂ (30 mL), (3-mercaptopropylsulfanyl) acetic acid methyl ester 4 (1.93 g, 10.7 mmol), prepared according to Chem. Pharm. Bull. 28 (2), 1980, 558-566, was added and the solution was cooled to 0° C. Basic alumina (11.9 g) was added and the reaction mixture was warmed to room temperature. After stirring for 18 h the mixture was filtered through celite and concentrated in vacuo. The residue was purified by flash column chromatography (silica gel, 6:1 hex/EtOAc) to yield 3.6 g (80%) of the above titled compound.

### EXAMPLE 4

### (3-{(1R,2S,3R)-3-(tert-Butyldimethylsilanyloxy)-2-[(S)-(E)-3-(tert-butyldimethylsilanoxy)oct-1-enyl]-5-oxocyclopentylsulfanyl}propylsulfanyl)acetic acid methyl ester (7)

tert-Butyllithium (1.47 mL of a 1.7M solution in pentane, 2.5 mmol) was added dropwise to a solution of tert-butyl[(S)-1-((E)-2-iodovinyl) hexyloxy]dimethylsilane 6 (462.5 mg, 1.25 mmol) in Et₂O (6.0 mL) at −78° C. After stirring for 30 min lithium 2-thienylcyanocuprate (6.0 mL of a 0.25M solution in THF, 1.5 mmol) was added and the reaction was stirred an additional 30 min at −78° C. A solution of enone 5 (430 mg, 1.1 mmol) in Et₂O (1 mL) was added and stirring was continued for an additional 1 h. The reaction mixture was then quickly poured into saturated aqueous NH₄Cl cooled to 0° C. The mixture was extracted with EtOAc and the organic portion was washed with brine, dried (Na₂SO₄), filtered and concentrated in vacuo. The residue was quickly purified by flash column chromatography (silica gel, 100% hexane followed by 8:1 hex/EtOAc) to afford 270 mg (39%) of the above titled compound.

### EXAMPLE 5

### {3-[(1R,2S,3R)-3-Hydroxy-2-((S)-(E)-3-hydroxyoct-1-enyl)-5-oxocyclopentyl-sulfanyl]propylsulfanyl}acetic acid methyl ester (8)

Hydrogen fluoride-pyridine (220 μL) was added to a solution of bis-TBDMS ether 7 (70 mg, 0.11 mmol) in CH₃CN (2.0 mL) at 0° C. The reaction was warmed to room temperature, stirred 1 h, and recooled to 0° C. The reaction was quenched with saturated aqueous NaHCO₃ until gas evolution ceased. The mixture was extracted with CH₂Cl₂ (4×). The combined organics were washed with brine, dried (Na₂SO₄), filtered and concentrated in vacuo. Purification of the residue by flash column chromatography (silica gel, 100% CH₂Cl₂ followed by 30:1 CH₂Cl₂:MeOH) provided 40 mg (90%) of the above titled compound.

### EXAMPLE 6

### {3-[(1R,2S,3R)-3-Hydroxy-2-((S)-(E)-3-hydroxyoct-1-enyl)-5-oxocyclopentylsulfanyl]propylsulfanyl}acetic acid (9)

Methyl ester 8 (50 mg, 0.124 mmol) was dissolved in CH₃CN (10 mL) and pH 7.2 phosphate buffer (3.0 mL) was added. The mixture was treated with PLE (400 μL, 1.34 mol/L) and stirred for 16 h at 23° C. The reaction mixture was extracted with EtOAc (3×). The combined organics were washed with brine, dried (Na_{2 SO}₄), filtered and concentrated in vacuo. Purification of the residue by flash column chromatography (silica gel, 100% EtOAc) gave 5.3 mg (11%) of the above titled compound.

### EXAMPLE 7

### {3-[(1R,2S,3R)-3-Hydroxy-2-((S)-(E)-3-hydroxyoct-1-enyl)-5-oxocyclopentyl-sulfanyl]propylsulfanyl}acetic acid isopropyl ester (10)

Isopropyl-p-tolyltriazene (200 μL) was added dropwise to a solution of carboxylic acid 9 (10.5 mg, 0.026 mmol) in acetone (5.0 mL) at 23° C. After stirring for 1 h the reaction was quenched with 1N HCl and the solvent was removed in vacuo. The residue was extracted with CH₂Cl₂ (2×). The combined organics were dried (Na₂SO₄), filtered and concentrated in vacuo. Purification of the residue by flash column chromatography (silica gel, 4:1 hex/EtOAc) gave 4.3 mg (38%) of the above titled compound.

### EXAMPLE 8

### (3-{(1R,2S,3R)-3-(tert-Butyldimethylsilanyloxy)-2-[(S)-(E)-3-(tert-butyldimethylsilanoxy)-5-(naphthyl)pent-1-enyl]-5-oxocyclopentylsulfanyl}propylsulfanyl)acetic acid methyl ester (H)

### (3-{(1R,2S,3R)-3-(tert-Butyldimethylsilanyloxy)-2-[(S)-(E)-3-(tert-butyldimethylsilanoxy)-5-(naphthyl)pent-1-enyl]-5-oxocyclopentylsulfanyl}propylsulfanyl)acetic acid methyl ester (L)

The named compound is prepared by substituting tert-butyl-[(E)-3-iodo-1-(2-naphthalen-2-yl-ethyl) allyloxy]dimethylsilane for tert-butyl[(S)-1-((E)-2-iodovinyl)hexyloxy]dimethylsilane in the method of Example 4. FCC gives a higher Rf compound and a lower Rf compound, designated as H and L, respectively.

### EXAMPLE 9(H)

### {3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid methyl ester (H)

The named compound is prepared by repeating the method of Example 5 with the named compound of Example 8 (H) rather then the named compound of Example 4.

### EXAMPLE 9 (L)

### {3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid methyl ester (L)

The named compound is prepared by repeating the method of Example 5 with the named compound of Example 8 (L) rather then the named compound of Example 4.

### EXAMPLE 10 (H)

### {3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid (H)

The named compound is prepared by repeating the method of Example 6 with the named compound of Example 9 (H) rather than the named compound of Example 5.

### EXAMPLE 10 (L)

### {3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid (L)

The named compound is prepared by repeating the method of Example 6 with the named compound of Example 9 (L) rather than the named compound of Example 5.

### EXAMPLE 11

### {3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid isopropyl ester

The named compound is prepared by repeating the method of Example 7 with the named compound of Example 10 rather than the named compound of Example 6.

### EXAMPLE 12

### (3-{(1R,2S,3R)-3-(tert-Butyldimethylsilanyloxy)-2-[(S)-(E)-3-(tert-butyldimethylsilanoxy)-5-(benzothienyl)pent-1-enyl]-5-oxocyclopentylsulfanyl}propylsulfanyl)acetic acid methyl ester (H)

### (3-{(1R,2S,3R)-3-(tert-Butyldimethylsilanyloxy)-2-[(S)-(E)-3-(tert-butyldimethylsilanoxy)-5-(benzothienyl)pent-1-enyl]-5-oxocyclopentylsulfanyl}propylsulfanyl)acetic acid methyl ester (L)

The named compound is prepared by substituting [(E)-1-(2-Benzo[b]thiophen-2-yl-ethyl)-3-iodoallyloxy]-tert-butyldimethylsilane for tert-butyl [(S)-1-((E)-2-iodovinyl)hexyloxy]dimethylsilane in the method of Example 4. FCC gives a higher Rf compound and a lower Rf compound, designated as H and L, respectively.

### EXAMPLE 13(H)

### {3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid methyl ester (H)

The named compound is prepared by repeating the method of Example 5 with the named compound of Example 12 (H) rather then the named compound of Example 4.

### EXAMPLE 13(L)

### {3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid methyl ester (L)

The named compound is prepared by repeating the method of Example 5 with the named compound of Example 12 (H) rather then the named compound of Example 4.

### EXAMPLE 14(H)

### {3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid (H)

The named compound is prepared by repeating the method of Example 6 with the named compound of Example 13 (H) rather than the named compound of Example 5.

### EXAMPLE 14(L)

### {3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid (L)

The named compound is prepared by repeating the method of Example 6 with the named compound of Example 13 (L) rather than the named compound of Example 5.

### EXAMPLE 15

### {3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid isopropyl ester

The named compound is prepared by repeating the method of Example 7 with the named compound of Example 14 rather than the named compound of Example 6.

### EXAMPLE 16

### (3-{(1R,2S,3R)-3-(tert-Butyldimethylsilanyloxy)-2-[(S)-(E)-3-(tert-butyldimethylsilanoxy)-5-(benzofuranyl)pent-1-enyl]-5-oxocyclopentylsulfanyl}propylsulfanyl)acetic acid methyl ester

The named compound is prepared by substituting [(E)-1-(2-Benzo[b]furan-2-yl-ethyl)-3-iodoallyloxy]-tert-butyldimethylsilane for tert-butyl[(S)-1-((E)-2-iodovinyl) hexyloxy]dimethylsilane in the method of Example 4.

### EXAMPLE 17

### {3-[(1R,2S,3R)-3-Hydroxy-2-((S)-(E)-3-hydroxy-5-(benzofuranyl)pent-1-enyl)-5-oxocyclopentylsulfanyl]propylsulfanyl}acetic acid methyl ester

The named compound is prepared by repeating the method of Example 5 with the named compound of Example 16 rather then the named compound of Example 4.

### EXAMPLE 18

### {3-[(1R,2S,3R)-3-Hydroxy-2-((S)-(E)-3-hydroxy-5-(benzofuranyl)pent-1-enyl)-5-oxocyclopentylsulfanyl]propylsulfanyl}acetic acid

The named compound is prepared by repeating the method of Example 6 with the named compound of Example 17 rather than the named compound of Example 5.

### EXAMPLE 19

### {3-[(1R,2S,3R)-3-Hydroxy-2-((S)-(E)-3-hydroxy-5-(benzofuranyl)pent-1-enyl)-5-oxocyclopentylsulfanyl]propylsulfanyl}acetic acid isopropyl ester

The named compound is prepared by repeating the method of Example 7 with the named compound of Example 18 rather than the named compound of Example 6.

### EXAMPLE 20

### (3-{(1R,2S,3R)-3-(tert-Butyldimethylsilanyloxy)-2-[(E)-3-(tert-butyldimethylsilanoxy)₄-naphthalen-2-yl-but-1-enyl]-5-oxocyclopentylsulfanyl}propylsulfanyl)acetic acid methyl ester (H)

### (3-{(1R,2S,3R)-3-(tert-Butyldimethylsilanyloxy)-2-[(E)-3-(tert-butyldimethylsilanoxy)-4-naphthalen-2-yl-but-1-enyl]-5-oxocyclopentylsulfanyl}propylsulfanyl)acetic acid methyl ester (L)

The named compound is prepared by substituting tert-butyl-((E)-3-iodo-1-naphthalen-2-yl-methylallyloxy)dimethylsilane for tert-butyl[(S)-1-((E)-2-iodovinyl)hexyloxy]dimethylsilane in the method of Example 4. FCC gives a higher Rf compound and a lower Rf compound, designated as H and L, respectively.

### EXAMPLE 21 (H)

### {3-[(1R,2S,3R)-3-Hydroxy-2-((E)-3-hydroxy-4-naphthalen-2-yl-but-1-enyl)-5-oxocyclopentylsulfanyl]propylsulfanyl}acetic acid methyl ester (H)

The named compound is prepared by repeating the method of Example 5 with the named compound of Example 20 (H) rather then the named compound of Example 4.

### EXAMPLE 21(L)

### {3-[(1R,2S,3R)-3-Hydroxy-2-((E)-3-hydroxy-4-naphthalen-2-yl-but-1-enyl)-5-oxocyclopentylsulfanyl]propylsulfanyl}acetic acid methyl ester (L)

The named compound is prepared by repeating the method of Example 5 with the named compound of Example 20 (H) rather then the named compound of Example 4.

### EXAMPLE 22(H)

### {3-[(1R,2S,3R)-3-Hydroxy-2-((E)-3-hydroxy-4-naphthalen-2-yl-but-1-enyl)-5-oxocyclopentylsulfanyl]propylsulfanyl}acetic acid (H)

The named compound is prepared by repeating the method of Example 6 with the named compound of Example 21 (H) rather than the named compound of Example 5.

### EXAMPLE 22(L)

### {3-[(1R,2S,3R)-3-Hydroxy-2-((E)-3-hydroxy-4-naphthalen-2-yl-but-1-enyl)-5-oxocyclopentylsulfanyl]propylsulfanyl}acetic acid (L)

The named compound is prepared by repeating the method of Example 6 with the named compound of Example 21 (H) rather than the named compound of Example 5.

### EXAMPLE 23

### {3-[(1R,2S,3R)-2-[(E)-4-Benzo[b]thiophen-3-yl-3-(tert-butyldimethylsilanyloxy)but-1-enyl]-3-(tert-butyldimethylsilanyloxy)-5-oxocyclopentylsulfanyl]propylsulfanyl}acetic acid methyl ester (H)

### {3-[(1R,2S,3R)-2-[(E)-4-Benzo[b]thiophen-3-yl-3-(tert-butyldimethylsilanyloxy)but-1-enyl]-3-(tert-butyldimethylsilanyloxy)-5-oxocyclopentylsulfanyl]propylsulfanyl}acetic acid methyl ester (L)

The named compound is prepared by substituting ((E)-1-Benzo[b]thiophen-3-ylmethyl-3-iodo-allyloxy)-tert-butyldimethylsilane for tert-butyl[(S)-1-((E)-2-iodovinyl)hexyloxy]dimethylsilane in the method of Example 4. FCC gives a higher Rf compound and a lower Rf compound, designated as H and L respectively.

### EXAMPLE 24(H)

### {3-[(1R,2S,3R)-2-((E)-4-Benzo[b]thiophen-3-yl-3-hydroxybut-1-enyl)-3-hydroxy-5-oxocyclopentylsulfanyl]propylsulfanyl}acetic acid methyl ester (H)

The named compound is prepared by repeating the method of Example 5 with the named compound of Example 23 (H) rather then the named compound of Example 4.

### EXAMPLE 24(L)

### {3-[(1R,2S,3R)-2-((E)-4-Benzo[b]thiophen-3-yl-3-hydroxybut-1-enyl)-3-hydroxy-5-oxocyclopentylsulfanyl]propylsulfanyl}acetic acid methyl ester (L)

The named compound is prepared by repeating the method of Example 5 with the named compound of Example 23 (H) rather then the named compound of Example 4.

### EXAMPLE 25(H)

### {3-[(1R,2S,3R)-2-((E)-4-Benzo[b]thiophen-3-yl-3-hydroxybut-1-enyl)-3-hydroxy-5-oxocyclopentylsulfanyl]propylsulfanyl}acetic acid (H)

The named compound is prepared by repeating the method of Example 6 with the named compound of Example 24 (H) rather than the named compound of Example 5.

### EXAMPLE 25(L)

### {3-[(1R,2S,3R)-2-((E)-4-Benzo[b]thiophen-3-yl-3-hydroxybut-1-enyl)-3-hydroxy-5-oxocyclopentylsulfanyl]propylsulfanyl}acetic acid (L)

The named compound is prepared by repeating the method of Example 6 with the named compound of Example 24 (H) rather than the named compound of Example 5.

### EXAMPLE 26

### (3-{(1R,2S,3R)-3-(tert-Butyldimethylsilanyloxy)-2-[(S)-(E)-3-(tert-butyldimethylsilanoxy)-3-(methyl)-5-(naphthyl)pent-1-enyl]-5-oxocyclopentylsulfanyl}propylsulfanyl)acetic acid methyl ester (H)

### (3-{(1R,2S,3R)-3-(tert-Butyldimethylsilanyloxy)-2-[(S)-(E)-3-(tert-butyldimethylsilanoxy)-3-(methyl)-5-(naphthyl)pent-1-enyl]-5-oxocyclopentylsulfanyl}propylsulfanyl)acetic acid methyl ester (L)

The named compound is prepared by substituting tert-Butyl-[(E)-3-iodo-1-methyl-1-(2-naphthalen-2-yl-ethyl) allyloxy]dimethylsilane for tert-butyl [(S)-1-((E)-2-iodovinyl)hexyloxy]dimethylsilane in the method of Example 4. FCC gives a higher Rf compound and a lower Rf compound, designated as H and L, respectively.

### EXAMPLE 27(H)

### {3-[(1R,2S,3R)-3-Hydroxy-2-((S)-(E)-3-(hydroxy)-3-(methyl)-5-(naphthyl)pent-1-enyl)-5-oxocyclopentylsulfanyl]propylsulfanyl}acetic acid methyl ester (H)

The named compound is prepared by repeating the method of Example 5 with the named compound of Example 26 (H) rather then the named compound of Example 4.

### EXAMPLE 27(L)

### {3-[(1R,2S,3R)-3-Hydroxy-2-((S)-(E)-3-(hydroxy)-3-(methyl)-5-(naphthyl)pent-1-enyl)-5-oxocyclopentylsulfanyl]propylsulfanyl}acetic acid methyl ester (L)

The named compound is prepared by repeating the method of Example 5 with the named compound of Example 26 (H) rather then the named compound of Example 4.

### EXAMPLE 28(H)

### {3-[(1R,2S,3R)-3-Hydroxy-2-((S)-(E)-3-(hydroxy)-3-(methyl)-5-(naphthyl)pent-1-enyl)-5-oxocyclopentylsulfanyl]propylsulfanyl}acetic acid (H)

The named compound is prepared by repeating the method of Example 6 with the named compound of Example 27 (H) rather than the named compound of Example 5.

### EXAMPLE 28(L)

### {3-[(1R,2S,3R)-3-Hydroxy-2-((S)-(E)-3-(hydroxy)-3-(methyl)-5-(naphthyl)pent-1-enyl)-5-oxocyclopentylsulfanyl]propylsulfanyl}acetic acid (L)

The named compound is prepared by repeating the method of Example 6 with the named compound of Example 27(L) rather than the named compound of Example 5.

### EXAMPLE 29

### (3-{(1R,2S,3R)-3-(tert-Butyldimethylsilanyloxy)-2-[(E)-3-(tert-butyldimethylsilanoxy)-3-methyl-4-naphthalen-2-yl-but-1-enyl]-5-oxocyclopentylsulfanyl}propylsulfanyl)acetic acid methyl ester (H)

### (3-{(1R,2S,3R)-3-(tert-Butyldimethylsilanyloxy)-2-[(E)-3-(tert-butyldimethylsilanoxy)-3-methyl-4-naphthalen-2-yl-but-1-enyl]-5-oxocyclopentylsulfanyl}propylsulfanyl)acetic acid methyl ester (L)

The named compound is prepared by substituting tert-butyl-[(E)-3-iodo-1-methyl-1-(2-naphthalen-2-yl-methyl)allyloxy]dimethylsilane for tert-butyl[(S)-1-((E)-2-iodovinyl) hexyloxy]dimethylsilane in the method of Example 4. FCC gives a higher Rf compound and a lower Rf compound, designated as H and L, respectively.

### EXAMPLE 30(H)

### {3-[(1R,2S,3R)-3-Hydroxy-2-((E)-3-hydroxy-3-methyl-4-naphthalen-2-yl-but-1-enyl)-5-oxocyclopentylsulfanyl]propylsulfanyl}acetic acid methyl ester (H)

The named compound is prepared by repeating the method of Example 5 with the named compound of Example 29 (H) rather then the named compound of Example 4.

### EXAMPLE 30(L)

### {3-[(1R,2S,3R)-3-Hydroxy-2-((E)-3-hydroxy-3-methyl-4-naphthalen-2-yl-but-1-enyl)-5-oxocyclopentylsulfanyl]propylsulfanyl}acetic acid methyl ester (L)

The named compound is prepared by repeating the method of Example 5 with the named compound of Example 29 (L) rather then the named compound of Example 4.

### EXAMPLE 31(H)

### {3-[(1R,2S,3R)-3-Hydroxy-2-((E)-3-hydroxy-3-methyl-4-naphthalen-2-yl-but-1-enyl)-5-oxocyclopentylsulfanyl]propylsulfanyl}acetic acid (H)

The named compound is prepared by repeating the method of Example 6 with the named compound of Example 30 (H) rather than the named compound of Example 5.

### EXAMPLE 31(L)

### {3-[(1R,2S,3R)-3-Hydroxy-2-((E)-3-hydroxy-3-methyl-4-naphthalen-2-yl-but-1-enyl)-5-oxocyclopentylsulfanyl]propylsulfanyl}acetic acid (L)

The named compound is prepared by repeating the method of Example 6 with the named compound of Example 30 (L) rather than the named compound of Example 5.

### EXAMPLE 32

### (3-{(1R,2S,3R)-3-(tert-Butyldimethylsilanyloxy)-2-[(S)-(E)-3-(tert-butyldimethylsilanoxy)-3-(methyl)-5-(benzothienyl)pent-1-enyl]-5-oxocyclopentylsulfanyl}propylsulfanyl)acetic acid methyl ester (H)

### (3-{(1R,2S,3R)-3-(tert-Butyldimethylsilanyloxy)-2-[(S)-(E)-3-(tert-butyldimethylsilanoxy)-3-(methyl)-5-(benzothienyl)pent-1-enyl]-5-oxocyclopentylsulfanyl}propylsulfanyl)acetic acid methyl ester (L)

The named compound is prepared by [(E)-1-(2-Benzo[b]thiophen-2-yl-ethyl)-3-iodo-1-methylallyloxy]-tert-butyldimethylsilane for tert-butyl [(S)-1-((E)-2-iodovinyl) hexyloxy]dimethylsilane in the method of Example 4. FCC gives a higher Rf compound and a lower Rf compound, designated as H and L, respectively.

### EXAMPLE 33(H)

### {3-[(1R,2S,3R)-3-Hydroxy-2-((S)-(E)-3-(hydroxy)-3-(methyl)-5-(benzothienyl)pent-1-enyl)-5-oxocyclopentylsulfanyl]propylsulfanyl}acetic acid methyl ester (H)

The named compound is prepared by repeating the method of Example 5 with the named compound of Example 32 (H) rather then the named compound of Example 4.

### EXAMPLE 33(L)

### {3-[(1R,2S,3R)-3-Hydroxy-2-((S)-(E)-3-(hydroxy)-3-(methyl)-5-(benzothienyl)pent-1-enyl)-5-oxocyclopentylsulfanyl]propylsulfanyl}acetic acid methyl ester (L)

The named compound is prepared by repeating the method of Example 5 with the named compound of Example 32 (L) rather then the named compound of Example 4.

### EXAMPLE 34(H)

### {3-[(1R,2S,3R)-3-Hydroxy-2-((S)-(E)-3-(hydroxy)-3-(methyl)-5-(benzothienyl)pent-1-enyl)-5-oxocyclopentylsulfanyl]propylsulfanyl}acetic acid (H)

The named compound is prepared by repeating the method of Example 6 with the named compound of Example 33 (H) rather than the named compound of Example 5.

### EXAMPLE 34(L)

### {3-[(1R,2S,3R)-3-Hydroxy-2-((S)-(E)-3-(hydroxy)-3-(methyl)-5-(benzothienyl)pent-1-enyl)-5-oxocyclopentylsulfanyl]propylsulfanyl}acetic acid (L)

The named compound is prepared by repeating the method of Example 6 with the named compound of Example 33L rather than the named compound of Example 5.

The compounds of the Examples are subject to in vitro testing as described below. The results are reported in the table as IC50s in nM.

| Example No. | | hEP₂ | hEP₃ | hEP₄ |
|---|---|---|---|---|
| 33H | 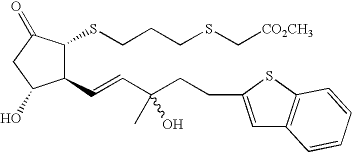 | NA | NA | 200 |
| 33L | 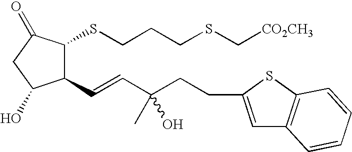 | NA | NA | 300 |
| 34H | 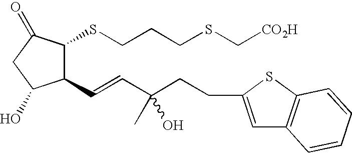 | >>10⁴ | >10⁴ | 32 |
| 34L | 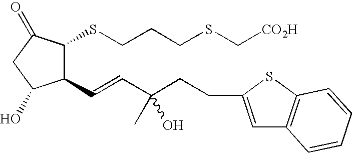 | NA | >10⁴ | 68 |
| 13H | 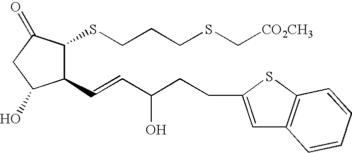 | NA | NA | 91 |
| 13L | 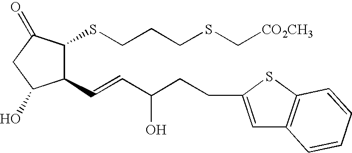 | >>10⁴ | 7200 | 93 |
| 14H | 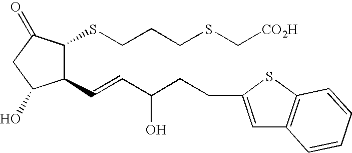 | >>10⁴ | >10⁴ | 27 |
| 14L | 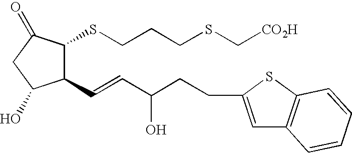 | 10⁴ | >10⁴ | 13 |
| 9H | 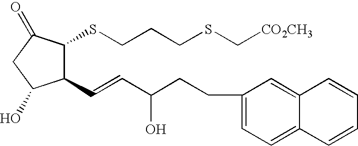 | NA | NA | 40 |
| 9L | 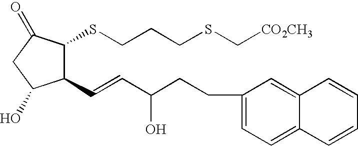 | NA | >10⁴ | 40 |
| 10H | 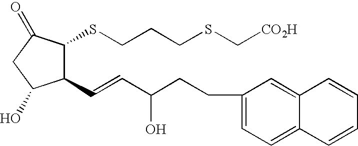 | >>10⁴ | >10⁴ | 450 |
| 10L | 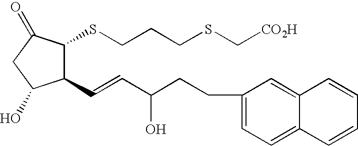 | >10⁴ | 8300 | 19.5 |
| 27H | 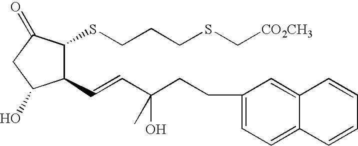 | NA | NA | 500 |
| 27L | 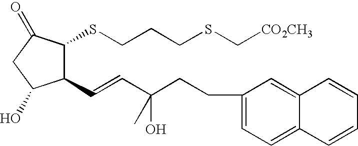 | NA | NA | 3400 |
| 28H | 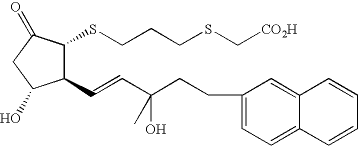 | NA | >10⁴ | 1700 |
| 28L | 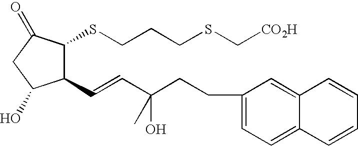 | NA | >10⁴ | 1500 |
| 21H | 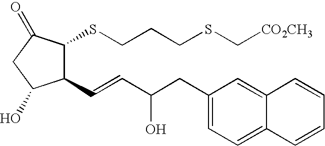 | NA | >10⁴ | 100 |
| 21L | 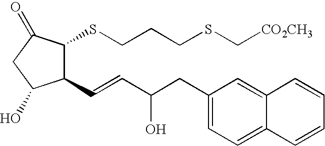 | NA | >10⁴ | 13 |
| 22H | 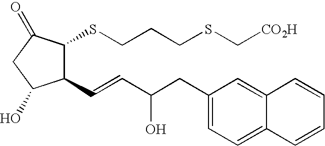 | NA | >10⁴ | 32 |
| 22L | 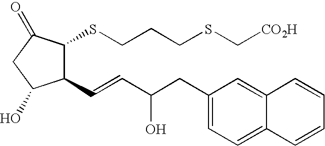 | >>10⁴ | >10⁴ | 6.2 |
| 30H | 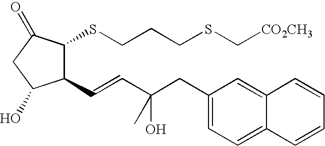 | NA | >10⁴ | 3100 |
| 30L | 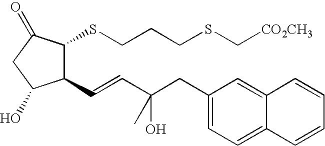 | NA | NA | 3200 |
| 31H | 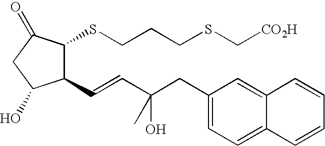 | NA | 8100 | 300 |
| 31L | 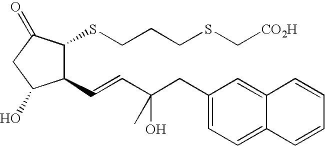 | NA | 9300 | 900 |
| 24H | 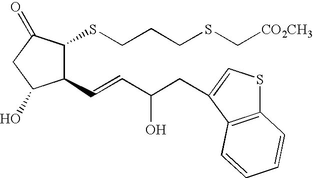 | NA | NA | 200 |
| 24L | 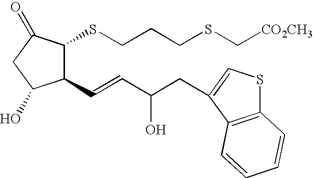 | 9300 | >10⁴ | 30 |
| 25H | 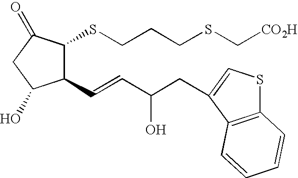 | >10⁴ | NA | 69 |
| 25L | 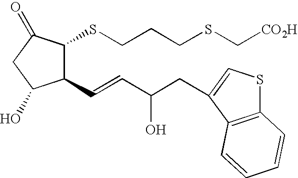 | 2200 | >10⁴ | 5 |

Human Recombinant EP₁, EP₂, EP₄, and FP Receptors: Stable Transfectants.

HEK-293 cells stably expressing the human or feline FP receptor, or EP₁, EP₂, EP₃, or EP₄ receptors were washed with TME buffer, scraped from the bottom of the flasks, and homogenized for 30 sec using a Brinkman PT 10/35 polytron. TME buffer was added to achieve a final 40 ml volume in the centrifuge tubes (the composition of TME is 100 mM TRIS base, 20 mM MgCl₂, 2M EDTA; 10N HCl is added to achieve a pH of 7.4).

The cell homogenate was centrifuged at 19000 r.p.m. for 20 min at 4° C. using a Beckman Ti-60 rotor. The resultant pellet was resuspended in TME buffer to give a final 1 mg/ml protein concentration, as determined by Biorad assay. Radioligand binding competition assays vs. [³H-]17-phenyl PGF_{2α}, (5 nM) were performed in a 100 μl volume for 60 min. Binding reactions were started by adding plasma membrane fraction. The reaction was terminated by the addition of 4 ml ice-cold TRIs-HCl buffer and rapid filtration through glass fiber GF/B filters using a Brandel cell harvester. The filters were washed 3 times with ice-cold buffer and oven dried for one hour. Non-specific binding was determined with 10 uM unlabeled 17-phenyl PGF_{2α}.

[³H-] PGE₂ (5 nM; specific activity 180 Ci mmol) was used as the radioligand for EP receptors. Binding studies employing EP₁, EP₂, EP₃, EP₄ were performed in duplicate in at least three separate experiments. A 200 μl assay volume was used. Incubations were for 60 min at 25° C. and were terminated by the addition of 4 ml of ice-cold 50 mM TRIs-HCl, followed by rapid filtration through Whatman GF/B filters and three additional 4 ml washes in a cell harvester (Brandel). Non-specific binding determined with 10⁻⁵M of unlabeled PGE₂.

Plasmids encoding the human EP₁, EP₂, EP₄, and FP receptors were prepared by cloning the respective coding sequences into the eukaryotic expression vector pCEP4 (Invitrogen). The pCEP4 vector contains an Epstein Barr virus (EBV) origin of replication, which permits episomal replication in primate cell lines expressing EBV nuclear antigen (EBNA-1). Similarly, competitive experiments were caried out using [3H]17-phenylPGF_{2α} at 5 nM in the presence of test ligands at various concentrations. Also non-specific binding was determined in the presence of excess unlabeled PGF_{2α} (10⁻⁵ M).

It also contains a hygromycin resistance gene that is used for eukaryotic selection. The cells employed for stable transfection were human embryonic kidney cells (HEK-293) that were transfected with and express the EBNA-1 protein. These HEK-293-EBNA cells (Invitrogen) were grown in medium containing Geneticin (G418) to maintain expression of the EBNA-1 protein. HEK-293 cells were grown in DMEM with 10% fetal bovine serum (FBS), 250 μg ml⁻¹ G418 (Life Technologies) and 200 μg ml⁻¹ gentamicin or penicillin/streptomycin. Selection of stable transfectants was achieved with 200 μg ml⁻¹ hygromycin, the optimal concentration being determined by previous hygromycin kill curve studies.

For transfection, the cells were grown to 50-60% confluency on 10 cm plates. The plasmid pCEP4 incorporating cDNA inserts for the respective human prostanoid receptor (20 μg) was added to 500 μl of 250 mM CaCl₂. HEPES buffered saline×2 (2×HBS, 280 mM NaCl, 20 mM HEPES acid, 1.5 mM Na₂HPO₄, pH 7.05-7.12) was then added dropwise to a total of 500 μl, with continuous vortexing at room temperature. After 30 min, 9 ml DMEM were added to the mixture. The DNA/DMEM/calcium phosphate mixture was then added to the cells, which had been previously rinsed with 10 ml PBS. The cells were then incubated for 5 hr at 37° C. in humidified 95% air/5% CO₂. The calcium phosphate solution was then removed and the cells were treated with 10% glycerol in DMEM for 2 min. The glycerol solution was then replaced by DMEM with 10% FBS. The cells were incubated overnight and the medium was replaced by DMEM/10% FBS containing 250 μg ml⁻¹ G418 and penicillin/streptomycin. The following day hygromycin B was added to a final concentration of 200 μg ml⁻¹.

Ten days after transfection, hygromycin B resistant clones were individually selected and transferred to a separate well on a 24 well plate. At confluence each clone was transferred to one well of a 6 well plate, and then expanded in a 10 cm dish. Cells were maintained under continuous hygromycin selection until use.

Human Recombinant EP₃ and TP Receptors: Transient Transfectants.

Plasmids encoding the human EP₃ (D isoform) or TP receptor were prepared by cloning the respective coding sequences into a pcDNA₃ vector (Invitrogen). COS-7 cells were transfected with pcDNA₃ containing cDNA encoding the EP₃ or TP receptor by employing the lipofectin method, according to the manufacturers instructions (Gibco). For radioligand binding studies, cells were harvested two days after transfection.

Radioligand Binding

Radioligand binding studies on plasma membrane fractions prepared from cells were performed as follows. Cells washed with TME buffer were scraped from the bottom of the plates and homogenized for 30 sec using a Brinkman PT 10/35 polytron. TME buffer was added as necessary to achieve a 40 ml volume in the centrifuge tubes. TME is comprised of 50 mM TRIS base, 10 mM MgCl₂, 1 mM EDTA; pH 7.4 is achieved by adding 1 N HCl. The cell homogenate was centrifuged at 19,000 rpm for 20-25 min at 4° C. using a Beckman Ti-60 or Tt-70 rotor. The pellet was then resuspended in TME buffer to provide a final protein concentration of 1 mg/ml, as determined by Bio-Rad assay. Radioligand binding assays were performed in a 100 μl or 200 μl volume.

The binding of [³H] PGE₂ (specific activity 165 Ci/mmol) was determined in duplicate and in at least 3 separate experiments. Incubations were for 60 min at 25° C. and were terminated by the addition of 4 ml of ice-cold 50 mM TRIS-HCl followed by rapid filtration through Whatman GF/B filters and three additional 4 ml washes in a cell harvester (Brandel). Competition studies were performed using a final concentration of 2.5 or 5 nM [³H] PGE₂ and non-specific binding was determined with 10⁻⁵ M unlabelled PGE₂.

The binding of [³H]-SQ29548 (specific activity 41.5 Ci mmol⁻¹) at TP receptors were determined in duplicate in at least three separate experiments. Radiolabeled SQ29548 was purchased from New England Nuclear. Incubations were for 60 min at 25° C. and were terminated by the addition of 4 ml of ice-cold 50 mM TRIs-HCl, followed by rapid filtration through Whatman GF/B filters and three additional 4 ml washes in a cell harvester (Brandel). Competition studies were performed using a final concentration of 10 nM [³H]-SQ 29548 and non-specific binding determined with 10 μM of the unlabeled prostanoid. For all radioligand binding studies, the criteria for inclusion were >50% specific binding and between 500 and 1000 displaceable counts or better.

While not intending to limit the scope of the invention in any way, the results of the in vitro tests suggest that the compounds described herein are selective EP4 agonists, and will thus be useful for the treatment of inflammatory bowel disease.

The foregoing description details specific methods and compositions that can be employed to practice the present invention, and represents the best mode contemplated. However, it is apparent for one of ordinary skill in the art that further compounds with the desired pharmacological properties can be prepared in an analogous manner, and that the disclosed compounds can also be obtained from different starting compounds via different chemical reactions. Similarly, different pharmaceutical compositions may be prepared and used with substantially the same result. Thus, however detailed the foregoing may appear in text, it should not be construed as limiting the overall scope hereof; rather, the ambit of the present invention is to be governed only by the lawful construction of the appended claims.

1. A method comprising administering a compound to a mammal suffering from an inflammatory bowel disease for the treatment of said disease, said compound represented by the general formula I; 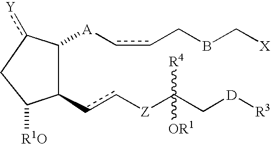
wherein hatched lines represent the α configuration, a triangle represents the β configuration, a wavy line represents either the α configuration or the β configuration and a dotted line represents the presence or absence of a double bond;
A and B are independently selected from the group consisting of O, S and CH₂, provided that at least one of A or B is S;
D represents a covalent bond or CH₂, O, S or NH;
X is CO₂R, CONR₂, CH₂OR, P(O)(OR)₂, CONRSO₂R, SONR₂ or 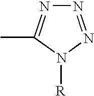
Y is O, OH, OCOR², halogen or cyano;
Z is CH₂ or a covalent bond;
R is H or R²;
R¹ is H, R², phenyl, or COR²;
R² is C₁-C₅ lower alkyl or alkenyl;
R₃ is benzothienyl, benzofuranyl, naphthyl, or substituted derivatives thereof, wherein the substituents maybe selected from the group consisting of C₁-C₅ alkyl, halogen, CF₃, CN, NO₂, NR₂, CO₂R and OR and R⁴ is hydrogen or C₁-C₅ lower alkyl.

2. The method according to claim 1 wherein said compound is represented by the general formula II;

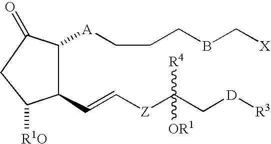

3. The method according to claim 2 wherein said compound is represented by the general formula III;

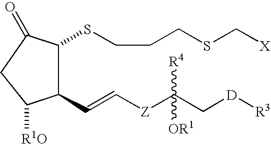

4. The method of claim 1 wherein Z represents a covalent bond.

5. The method of claim 1 wherein D represents a covalent bond or is CH₂.

6. The method of claim 1 wherein X is CO₂R.

7. The method of claim 6 wherein R is selected from the group consisting of H, methyl, i-propyl, and n-propenyl.

8. The method of claim 1 wherein R is H, or n-propenyl.

9. The method of claim 1 wherein R₁ is H.

10. The method of claim 1 wherein D is CH₂.

11. The method of claim 10 wherein R³ is benzo[b]thienyl, 3-chlorobenzo[b]thienyl or naphthyl.

12. The method of claim 1 wherein said compound is selected from the group consisting of
{3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid methyl ester,
{3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid,
{3-[(1R,2S,3R)-3-Hydroxy-2-((S)-(E)-3-(hydroxy)-5-(naphthyl)pent-1-enyl)-5-oxocyclopentylsulfanyl]propylsulfanyl}acetic acid isopropyl ester,
{3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid methyl ester,
{3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid,
{3-[(1R,2S,3R)-3-Hydroxy-2-((S)-(E)-3-hydroxy-5-(benzothienyl)pent-1-enyl)-5-oxocyclopentylsulfanyl]propylsulfanyl}acetic acid isopropyl ester,
{3-[(1R,2S,3R)-3-Hydroxy-2-((S)-(E)-3-hydroxy-5-(benzofuranyl)pent-1-enyl)-5-oxocyclopentylsulfanyl]propylsulfanyl}acetic acid methyl ester,
{3-[(1R,2S,3R)-3-Hydroxy-2-((S)-(E)-3-hydroxy-5-(benzofuranyl)pent-1-enyl)-5-oxocyclopentylsulfanyl]propylsulfanyl}acetic acid,
{3-[(1R,2S,3R)-3-Hydroxy-2-((S)-(E)-3-hydroxy-5-(benzofuranyl)pent-1-enyl)-5-oxocyclopentylsulfanyl]propylsulfanyl}acetic acid isopropyl ester,
{3-[(1R,2S,3R)-3-Hydroxy-2-((E)-3-hydroxy-4-naphthalen-2-yl-but-1-enyl)-5-oxocyclopentylsulfanyl]propylsulfanyl}acetic acid methyl ester,
{3-[(1R,2S,3R)-3-Hydroxy-2-((E)-3-hydroxy-4-naphthalen-2-yl-but-1-enyl)-5-oxocyclopentylsulfanyl]propylsulfanyl}acetic acid,
{3-[(1R,2S,3R)-2-((E)-4-Benzo[b]thiophen-3-yl-3-hydroxybut-1-enyl)-3-hydroxy-5-oxocyclopentylsulfanyl]propylsulfanyl}acetic acid methyl ester,
{3-[(1R,2S,3R)-2-((E)-4-Benzo[b]thiophen-3-yl-3-hydroxybut-1-enyl)-3-hydroxy-5-oxocyclopentylsulfanyl]propylsulfanyl}acetic acid,
{3-[(1R,2S,3R)-3-Hydroxy-2-((S)-(E)-3-(hydroxy)-3-(methyl)-5-(naphthyl)pent-1-enyl)-5-oxocyclopentylsulfanyl]propylsulfanyl}acetic acid methyl ester,
{3-[(1R,2S,3R)-3-Hydroxy-2-((E)-3-hydroxy-3-methyl-4-naphthalen-2-yl-but-1-enyl)-5-oxocyclopentylsulfanyl]propylsulfanyl}acetic acid methyl ester,
{3-[(1R,2S,3R)-3-Hydroxy-2-((E)-3-hydroxy-3 methyl-4-naphthalen-2-yl-but-1-enyl)-5-oxocyclopentylsulfanyl]propylsulfanyl}acetic acid,
{3-[(1R,2S,3R)-3-Hydroxy-2-((S)-(E)-3-(hydroxy)-3-(methyl)-5-(naphthyl)but-1-enyl)-5-oxocyclopentylsulfanyl]propylsulfanyl}acetic acid,
{3-[(1R,2S,3R)-3-Hydroxy-2-((S)-(E)-3-(hydroxy)-3-(methyl)-5-(benzothienyl)pent-1-enyl)-5-oxocyclopentylsulfanyl]propylsulfanyl}acetic acid methyl ester and
{3-[(1R,2S,3R)-3-Hydroxy-2-((S)-(E)-3-(hydroxy)-3-(methyl)-5-(benzothienyl)pent-1-enyl)-5-oxocyclopentylsulfanyl]propylsulfanyl}acetic acid methyl ester.

13. The method of claim 1 wherein D is CH₂ and Z represents a covalent bond.

14. The method of claim 1 wherein R⁴ is hydrogen or methyl.

15. The method of claim 1 wherein R⁴ is hydrogen.

16. The method of claim 1 wherein said irritable bowel disease is Crohn's disease.

17. The method of claim 1 wherein said irritable bowel disease is ulcerative colitis.

18. An oral or rectal dosage form comprising a therapeutically effective amount of a compound represented by the general Formula 1 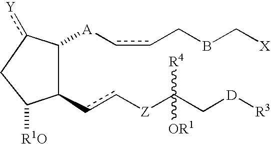
wherein hatched lines represent the α configuration, a triangle represents the β configuration, a wavy line represents the α configuration or the β configuration and a dotted line represents the presence or absence of a double bond;
A and B are independently selected from the group consisting of O, S and CH₂;
provided that at least one of A or B is S;
D represents a covalent bond or CH₂, O, S or NH;
X is CO₂R, CONR₂, CH₂OR, P(O)(OR)₂, CONRSO₂R SONR₂ or 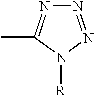
Y is O, OH, OCOR², halogen or group;
Z is CH₂ or a covalent bond;
R is H or R²;
R¹ is H, R², phenyl, or COR²;
R² is C₁-C₅ lower alkyl or alkenyl; and
R₃ is benzothienyl, benzofuranyl, naphthyl or substituted derivatives thereof, wherein the substituents maybe selected from the group consisting of C₁-C₅ alkyl, halogen, CF₃, CN, NO₂, NR₂, CO₂R and OR and R⁴ is hydrogen or C₁-C₅ alkyl.
